(19) United States
(12) Patent Application Publication
(10) Pub. No.: US 2013/0216585 A1
(43) Pub. Date: Aug. 22, 2013

(54) COMPOSITION AND METHODS FOR QUENCHING FREE RADICALS AND MODULATING INFLAMMATION

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/398,541

(22) Filed: Feb. 16, 2012

(51) Int. Cl. A61K 31/715; A61P 39/06; A61P 37/08; A61P 37/00; A61P 29/00; A61P 27/02

(52) U.S. Cl. 424/278.1

(57) ABSTRACT

Compositions and methods useful in quenching free radicals, the compositions including dihydroquercetin, vitamin C and Arabinogalactan, specifically, larch arabinogalactan, effective to enhance the immune modulating properties of Larch Arabinogalactan by quenching the free radicals that initiate a cascade of oxidative stress and inflammation.

### FIELD OF INVENTION

This invention and disclosure relates to compounds and methods that quench free radicals in a mammal, specifically a human, resulting in the inhibition of the upregulation of inflammatory cytokines by oxidative stress and resultant health benefits.

### BACKGROUND OF INVENTION

The relationship between oxidative stress and human health, particularly in regards to circulatory function and the risk of malignancy is now well accepted science. Harman, D, 1067 Ann N Y Acad Sci 10-21 (2006). All aerobic organisms require oxygen. While a human can live for several weeks without food, and several days without water, he cannot live longer than a few minutes without oxygen. However, the same molecule that gives us life is also the source of the degenerative diseases of aging and the again a process itself which ultimately is responsible for our the degenerative diseases of aging and death. We need oxygen to stay alive but our aerobic oxygen utilization produces dangerous free radicals that are toxic to the organs, cells, and even the cellular organelles of our bodies, the cellular mitochondria, and the DNA and RNA of the nucleus and ribosomes.

Oxidative Stress

Thousands of published studies implicate free radicals in the development of degenerative disease and accelerated aging. Knight J A. 28(6) Ann Clin Lab Sci. (1998). Huang H, Manton K G. 91100-17 Front Biosci. (2004). Young people naturally produce the antioxidant enzymes superoxide dismutase (SOD), catalase, and glutathione peroxidase that reduce and neutralize the free radicals produced during normal physiological processes. Levels of SOD and other critical antioxidant enzymes decline with age, contributing to the scourge of age-related disease. Oxygen toxicity was first described in 1878, when laboratory animals were exposed to pure oxygen, and these deleterious effects were further established in 1899. When placed in hyperbaric chambers of pure oxygen beyond normobaric atmospheres of pressure, laboratory animals die within a matter of minutes. By contrast, when the pressure of pure oxygen is slowly increased over a period of days, the animals manage to survive, because their bodies have time to produce higher-than-normal levels of the inducible antioxidant enzymes that protect against oxygen toxicity. The lungs of animals exposed to higher-than-normal oxygen concentrations reveal Massive tissue damage caused by too much oxygen, a condition known as hyperoxia. Thus, at higher-than-normal concentrations, oxygen is a toxic element and at normobaric pressure allows us to live to fairly well-defined species-specific lifespans. Knight J A. 28(6) Ann Clin Lab Sci. (1998).

The first toxic side effects of oxygen in humans became apparent in the 19th and early 20th century with the first cases of high pressure breathing of air during deep sea diving where oxygen poisoning and nitrogen narcosis caused the death, paralysis or lifetime debilitation of divers. During the Battle of Britain in World War II, pilots breathing pure oxygen for several hours daily developed emphysema, scarred lungs, and the effects of greatly accelerated aging. In some cases, the pilots looked and acted like men who were more than three times their age. One of the most tragic episodes of oxygen toxicity occurred later in the 1940s, when newborn infants were placed in oxygen-rich incubators. Some of the newborns went blind because their eye tissue had not yet accumulated dietary antioxidant carotenoids to protect against the toxic effects of higher-than-normal oxygen concentrations. Reedy E A. 23(2) Neonatal New 31-8 (2004).

The various free radical theories of aging and the oxygen free radical theory of aging all postulate that aging and the degenerative disease of aging are caused primarily from the oxygen utilization of aerobic organisms. In the evolution of life on earth, plants developed ways to harness the: energy of the sun through photosynthesis by using carbon dioxide and they gave off oxygen as a toxic metabolic by product. The eventual evolution of animals with greatly increased mobility, however, required a much greater source of energy to travel through the oceans and roam the land, so they developed a way to directly respire the oxygen produced by fauna in an increasing oxygen atmosphere. Their cells developed mitochondria capable of reducing oxygen to water in a four step reduction process utilizing cytochrome oxidase. The oxygen molecule, although not particularly chemically reactive in itself, produces powerful chemical reactive intermediates during its reduction. The partially reduced forms of oxygen are eventually converted in a four step, four electron addition to produce water and the energy liberated from the oxygen reduction is placed in the universal storage molecule ATP. Approximately 80% of oxygen is reduced to water in mitochondria using cytochrome C oxidase, with only 1% electron (superoxide) leakage across transport stage I between the initial formation of the superoxide radical to the terminal cytochrome oxidase. Oxygen goes in one end and water comes out the other end fully reduced with few detectable free radical intermediates being formed. Unfortunately, 20% of oxygen reductions in aerobic organisms occur with single electron additions to the oxygen molecule where cyctochrome oxidase is not utilized. This generally occurs in the presence of the copper and iron-containing enzymes following the quantum rules of the Pauli Exclusion Principal, where a single electron addition to the oxygen atom must be made because both unpaired electrons in each of the oxygen atoms of the oxygen: molecule have identical parallel spins and more than one parallel electron addition at any time is forbidden by quantum mechanics.

A single electron addition to the oxygen molecule forms the superoxide radical in 100% stoichiometric yield. Superoxide is the first free radical formed in biological systems. A second electron addition to the oxygen molecule produces hydrogen peroxide, a three electron addition to the oxygen molecule produces hydroxyl radical, and a four electron addition reduces oxygen to water.

Inflammatory Cascade

As in the case of oxidative stress, the association between inflammation and the various diseases, particularly of aging, are well established, but the interconnection between these two destructive pathways has only been demonstrated relatively recently. It was first published in relation to the HIV epidemic that began in the 1980's, when the benefit of N-acetyl cysteine, a less toxic source of dietary cysteine, a direct precursor and rate-limiting factor in glutathione peroxidase synthesis, was discovered. Researchers demonstrated that HIV-infected individuals have decreased intracellular GSH levels in their circulating T cells. Since GSH is the first major small molecule intracellular defense against the production of ROS, the researchers hypothesized that the observed decrease of GSH is due to a chronic oxidative stress induced by continual exposure to elevated levels of inflammatory cytokines, Roederer. M, et al, 8(2) AIDS Res Hum Retroviruses 209-17 (1992). More recently, diabetes has been shown to be related to a significant imbalance between the production of reactive oxygen species and antioxidant defenses, a condition termed oxidative stress. (Helmut Seiss, 1990) leading to alterations in stress-signaling pathways and eventual end-organ damage, i.e., the beta cells on the Isle of Langerhans. Oxidative stress and metabolic inflammation upregulate the expression of pro-inflammatory cytokines, including tissue necrosis factor alpha, monocyte chemo-attractant protein-1 and interleukin-6, as well as activating stress-sensitive kinases, such as c-Jun N-terminal kinase (JNK), phosphokinase C isoforms, mitogen-activated protein kinase and inhibitor of kappa B kinase. Lamb, R E, Goldstein, B J, 62(7) Int J Clin Pract 1087-95 (2008).

Dihydroquercetin

Dihydroquercetin is a bioflavonoid. Flavonoids display a wide range of biochemical and pharmacological properties, with one of the most thoroughly characterized effects being chemopreventive activity, measured by the chemopreventive index as a marker for the screening of potential agents. Dihydroquercetin, also known as taxifolin, exhibits high detoxification ability but lower cytotoxicity in cells, representative of a high chemo-preventive index. It has been shown to modulate the expression of several genes, including those coding for detoxification enzymes, cycle regulatory proteins, growth factors, and DNA repair proteins. Recent gene microarray study results show that the phase II detoxification enzymes, NQO1 and GSTM1, are upregulated, while the phase I detoxification enzyme, CYP2E1, is down regulated in the presence of taxifolin. Lee, S B, et al, 30(6) Biol Pharm Bull 1074-1079 (2007). Measurement of cytotoxicity to human lung embryonic fibroblasts (TIG-1) and umbilical vein endothelial (HUVE) cells, was examined and Dihydroquercetin shown to be the least cytotoxic of the ten tested flavonoids, with a 50% lethal concentration of >300 micromoles in TIG-1 and >200 micromoles in HUVE cells. Matsuo, M, et al, 28(2) Biol Pharm Bull 253-9 (2005).

Dihydroquercetin is a powerful antioxidant and is one substance in foods that significantly decreases the adverse effects of radical oxygen species, and reactive nitrogen species. Valko, M et al, 39(1) Int J Biochem Cell Biol. 44-84 (2007); Kostyuk V A, Potapovich Al, 355(1). Arch Biochem Biophys 43-8 (1998); Soliman K F & Mazzio E A, 218(4) Proc Soc Exp Biol Med 390-7 (1998). It was discovered to be an essential molecular intermediate in flavonoid biosynthetic pathways responsible for quercetin, oligo proanthocyanidins (OPC), and catechin formation and its superiority as an anti-oxidant has been demonstrated repeatedly. Tiukavkina N A, Rulenko I A, Kolesnik I A, 6 Vopr Pitan. 12-5(1997); Teselkin, I O, et al, 41(3) Biofizika. 620-4(1996).

Dihydroquercetin protects cell membranes from free radical damage, improves the activity of capillaries and supports the recovery of blood microcirculation throughout the body. Oxidative stress is reduced at the cellular level, as shown, for instance in cultured renal cells. Areias, F M, et al, 62(1) Biochem Pharmacol. 111-8(2001).

Due to Dihyroquercetin's permeability into human cells including blood cells, Dihydroquercetin has demonstrated reliable protection at the cellular level from a variety of reactive free:radical species.

The plasma levels of Dihydroquercetin remain higher in the human body longer than ordinary quercetin, as Dihydroquercetin is not broken down by the body's enzymes to less active compounds as rapidly or quantitatively nearly to the extent to that of quercetin. In addition, it is 400 times less mutagenic than quercetin in bacterial mutagenicity tests; quercetin has a minimum mutagenic dose of 4 nml to 1626 nmol for DHQ. Jurado, et al., 6(4) Mutagenesis 289-95 (1991).

Dihydroquercetin has been shown to inhibit the production of luminol-dependent chemiluminescence (CLIum) by activated human neutrophils, ‘T Hart, Chem Biol Interact. 1990; 73(2-3):323-35, and to inhibit their adhesion during inflammation. Wang.,Y H. et al, 67(12) Biochem Pharmacol 2251-62 (2004). Dihydroquercetinis reported to protect phagocytic cells from silica induced injury. Kostyuk V A, Potapovich A. I.; 355(1) Arch Biochem Biophys 43-8 (1998): Dihydroquercetin has shown activity as an inhibitor of histamine release by mast cells. Bronner C., Landry Y., 16(3) Agents Actions 147-51 (1985).

The partition coefficients of both catechin and taxifolin in a biomimetic system (micelles) were determined, since these properties may also contribute to the antioxidant behavior of this type of compound. The log P values determined depend on the electrostatic interactions of the compounds with the differently charged micelles (the highest values were obtained for zwitterionic and cationic micelles). The pro-oxidant behavior of the compounds was assessed through the oxidation of 8-hydroxy-2′-deoxyguanosine, induced by a Fenton reaction, catalyzed by copper. The data obtained revealed that the flavonoids under study did not present pro-oxidant activity, in this particular system. Teixeiria, S, 39(8) Free Radic Biol Med 1099-108 (2005). Cardiovascular function is also supported by flavonoids and in particular by Dihyroquercetin. It has been shown to inhibit lipoxygenases, the enzymes that participate in the oxidation of lipids in fatty acid bilayers. Wheeler, E. L. and Berry, D. L., 7(1) Carcinogenesis 33-6(1986). In combination with Vitamin C,. Dihydroqercetin increases erythrocyte deformability and decreases their aggregation. Plotnikov, M B.,et al, 104(12) Zh Nevrol Psikhiatr im S S Korsakova 33-7 (2004). Improved resistance of microsomes to lipid peroxidation has also been demonstrated for dihyroquercetin. Kravchenko L V, Morozov S V, Tutel'van V. A., 136(6) Bull Exp Biol.: 572-5 (2003).

Dihydroquercetin is a:far more powerful antioxidant than vitamins A, C and E, as indicated by its comparative USDA ranking of the Oxygen Radical Absorbance Capacity (ORAC) and supported by other additional studies such as Cell-based Antioxidant Protection (CAP-e) assay against peroxyl radical, which is used to test whether natural products contain antioxidants capable of entering into and protecting live cells from oxidative damage. Thus, when any protective effect is seen in the CAP-e assay, it shows a biologically meaningful antioxidant protection by the product.

Peroxide radical relates to oxidative damage of fatty acids and other lipids, and demonstrates why radicals such as peroxide and hydroxyl radicals can cause so much more damage than one might have expected due to the propagation stage of free radical generation in lipids. According to the chemical reactions involved, major antioxidant capacity assays are essentially divided into two categories: (1) hydrogen atom transfer (HAT) reaction based assays and (2) single electron transfer (ET) reaction based assays.

The majority of HAT-based assays apply a competitive reaction scheme, in which antioxidant and substrate compete for thermally generated peroxyl radicals through the decomposition of azo compounds. ORAC and CAP-e can also be applied as an assays for the inhibition of induced low-density lipoprotein oxidation and other oxygen radical absorbances. ORAChydro and CAP-e test the capacity of dihydroquercetin generated hydrogen atom transfer (HAT) to compete for thermally generated peroxyl radicals and hydroxyl radicals. For the latter it is necessary to use the HORAC assay test. Unlike electron transfer based assays that measure an antioxidant's reducing capacity, and the HAT-based assays measure the hydrogen atom donating capacity. The hydrogen atom transfer is a key step in the radical chain reaction. Therefore, the HAT-based assays are more relevant to the radical chain-breaking antioxidant capacity.

Among all the HAT-based assays, ORAC and CAP-e adopted an AUC (area under curve) technique to quantify antioxidant capacity. The advantage of the AUC approach is that it applies equally well for both antioxidants that exhibit distinct lag phases and those samples that have no lag phases. This approach unifies the lag time method and initial rate method, and it is particularly useful for food samples, which often contain multiple ingredients and have complex reaction kinetics.

Therefore, the ORAC and CAP-e assays has been broadly applied in academics and the food and supplement industry as the method of choice to quantify antioxidant capacity. In fact, an antioxidant database has been generated applying the ORAC assay in combination with the total phenols assay. The ORAChydro value of our dihydroquercetin has been measured at is 3,890 micromole of Trolox equivalent per 412.3 mg capsule or 28,000 micromole Trolox equivalent per gram. The CAP-e value of our dihydroquercetin has been measured at is 9.9 CAP-e units per gram.

It is recognized that Siberian and Dahurian larch species, Larch species: L. dahurica L., L. gmelinii, L. sibirica ledeb., also known colloquially as the Siberian and Mongolian larch is an excellent source for commercially feasible market volumes of dihyroquercetin.

### Vitamin C

The biological functions of Vitamin C (ascorbic. acid) are based primarily on its ability to provide reducing equivalents for a variety of biochemical reactions. Because of its reducing power, Vitamin C can reduce most physiologically relevant reactive oxygen species. As such, Vitamin C functions primarily as a co-factor for reactions requiring a reduced iron or copper metalloenzyme and as a protective antioxidant that operates in the aqueous phases both via intra and extra cellular pathways.

Vitamin C is known to be an electron donor for eight human enzymes. Three of these enzymes participate in collagen hydroxylation; two are utilized in carnitine biosynthesis and three are utilized in hormone and amino acid biosynthesis. Thus vitamin C plays a role in iron absorption, collagen formation, bone growth and amino acid metabolism. Vitamin C is involved in the synthesis and modulation of some hormonal components of the nervous system and helps to maintain healthy teeth, gums, cartilage, muscles, blood vessels and a healthy immune system.

Hypochlorite is produced from hydrogen peroxide from immune responder cells that utilize into kill invading foreign proteins, such as viruses and bacteria, but hypochlorite also damages normal body proteins. To provide antioxidant protection, the Recommended Dietary Allowance (RDA) for adults for vitamin C is set at 75 mg/day for females and 90 mg/day for males. This intake should maintain near maximal neutrophil ascorbate concentrations with little urinary excretion.

Because smokers suffer increased oxidative stress and metabolic turnover of Vitamin C, their recommended intake is increased by approximately 35 mg/day. A formula that includes Vitamin C should conform to or exceed these recommendations, and ideally should include DihydroqUercetin as a synergist and a recycling agent for Vitamin C as demonstrated in human clinical trials. Both .nutraceuticals are mediated by high- and low-affinity transporters. Due to homeostatic regulation, the biological half-life of ascorbate varies widely from 8 to 40 days and is inversely related to the ascorbate body pool. A total body pool of less than 300 mg is associated with scurvy symptoms in humans, while maximum body pools are limited to about 2-4 g.

The combination of Dihydroquercetin and Vitamin C, known as Ascovertin in Russia, has been shown to improve cellular rheology indices including a decrease of aggregation and increase of erythrocyte deformability as well as decrease of indices of lipid peroxidation in erythrocyte membrane and blood plasma, in patients with vascular encephalopathy. Plotnikov, M B, 104(12) Zh Nevrol Psikhiatr Im S S Korsakova 33-7 (2004). It has also been shown to protect rat retinal cells from the damage due to ultraviolet light. Logvinos, S V, et al, 140(5) Bull Exp Biol Med. 578-81 (2005).

### Arabinogalactan

Many herbs with well established immune-enhancing properties, such as Echinacea purpurea, Baptisia tinctoria, Thuja occidentalis, Angelica acutiloba and Curcuma longa also contain significant amounts of arbinogalactans. Polysaccharides are often found in the medicinal herbs used for immune enhancement, including Echinacea and Astragalus.

Arabinogalactans are a class of long, densely branched polysaccharides with molecular weights ranging from 6,000 to 120,000 or more and are found in the cell walls of a wide variety of edible and non-edible woody plants. Arabinogalactan is a fine, dry, off-white powder with a mildly sweet taste that mixes well with liquids. This safe and effective phytochemical has FDA GRAS status approval for use as a dietary fiber, even at large doses, and as a food additive. 5(5)Altern Med Rev 463-6 (2000) (no authors listed). The only reported side effect is occasional bloating and flatulence in a small percentage of people who take it.

The wood of the Western larch tree (Larix occidentalis) provides a rich harvest of free arabinogalactan from its inner bark. Because arabinogalactans posess potent biological activity and immune-enhancing properties, this unique dietary fiber is receiving increased attention as a clinically useful nutraceutical agent.

Larch is used medicinally for the effects of these polysaccharides on the intestines and immune system. Larch Arabinogalactan is a non-digestible soluble dietary fiber that resists breakdown by enzymes and enters the large bowel intact where it is fermented by colonic bacteria. Many edible and inedible plants are rich sources of Arabinogalactan, including leek seeds, carrots, radishes, black gram beans, pears, maize, wheat, red wine, Italian ryegrass, tomatoes, ragweed, sorghum, bamboo grass and coconut meat and milk.

Arabinogalactans are most abundant in the Larch tree (Larix spp.) Larch Arabinogalactan can be extracted from either the Western larch (Larix occidentalis), or Siberian and Dahurian larch (Larch species: L. dahurica L., L. gmelinii, L. sibirica ledeb.), previously mentioned colloquially as the Siberian and Mongolian larch. The Western larch is most commonly used.

### Immune Modulation

Arabinogalactan supports healthy immune system functioning. Schepetkin, I A, 5(13-14) Int Immunophamacol 1783-99. (2005). Larch Arabinogalactan, in particular, increases the levels of beneficial immune cells such as white blood cells without stimulating pro-inflammatory immune cell production or activation. Kelly, G S, 4(2) Altera Med Rev 96-103 (1999). Larch Arabinogalactan decreases the production of lymphoid cells in bone marrow. Currier, N L, 10 (2-3) Phytomedicine: 145-53(2003). It is highly effective in promoting in vitro and in vivo macrophage proliferation and exhibits anti-inflammatory, anti-complement, and anti-allergic effects. Kim L S, Waters R F, Burkholder P M., 7(2) Altera Med Rev 138-49 (2002).

Several studies have shown that Larch Arabinocalactan has anti-cancer activity in animals. In a 1987 study reported in the Journal of Cancer Research and Clinical Oncology, researchers tested their theory that organ-specific lectins (which represent a type of protein) help determine where malignant cells will metastasize in animals. Injections of arabinogalactans were shown to effectively block these lectins in mice livers and therefore prevent the spread of this mouse cancer. Beuth, J., 113 (1) J Cancer Res Clin Oncol 51-5 (1987).

This study was replicated successfully by researchers in Cologne, Germany, and published in 1988. Beuth, J., 6(2) Clin Exp Metastasis 115-20 (1988). A similar study in Sweden in 1991 found that pre-treatment with arabinogalactans reduced the number of liver metastates and lengthened survival times in laboratory animals. Hagmar B., Ryd W., Skomedal H., 11(6) invasion Metastasis 348-55 (1991).

A review of Larch Arabinogalactan research published in the Alternative Medicine Review in 1999 reported that the substance can stimulate natural-killer-cell activity and enhance other aspects of immune function as well. Kelly, G S, 4(2) Ahem Med Rev 96-103 (1999).

Larch Arabinogalactan enhances immune response in part due to its ability to stimulate “natural killer” (NK) cell cytotoxicity, a functional marker for health. Reports in the medical literature link decreased NK cell activity to a variety of chronic diseases including chronic fatigue syndrome, viral hepatitis, HIV/AIDS, and autoimmune diseases such as multiple sclerosis. Modified citrus pectin has the same anti-metastatic mechanism of action in animals as Larch Arabinogalactan, but does not provide the immune-modulating effects.

In a well-controlled study, Larch Arabinogalactan induced an increased release of interferon gamma (IFN gamma), tumor necrosis factor alpha, interleukin-1 beta (IL-1 beta) and interleukin-6 (IL-6). This resulted in activating two powerful cells of the immune system: macrophages and NK cells. It was found that the IFN gamma was most responsible for the observed enhancement of NK cytotoxicity. Hauer J. Anderer F A., 36(4) Cancer Immunol immunother. 237-44 (1993).

Other studies show Larch Arabinogalactan supports a decrease in the frequency and severity of pediatric otitis media caused by gram negative rods, especially Escherichia coli and Klebsiella.

### Digestion

Digestive disorders are very common and affect a great number of the population. The typical American diet, which is low in fiber and high in protein and carbohydrate, is a factor in the prevalence of these digestive disorders. Low levels of short-chain fatty acids and elevated levels of ammonia are associated with this type of diet. Intake of fiber, particularly Larch Arabinogalactan, has been shown to be supportive in combating the detrimental effects caused by poor diet. Larch Arabinogalactan has been shown to increase short-chain fatty acids, decrease colonic ammonia levels, increase the numbers of beneficial bacteria in the colon, as well as improve the immune response. These favorable effects of Larch Arabinogalactan have a positive modulation of many of these too-common intestinal factors.

Intestinal tracts are exposed to many substances—from antibiotics to protozoal parasites to sugary, processed foodsthat create an unfavorable atmosphere in the colon. The result can be constipation, diarrhea, candidiasis, parasitical infections and other conditions attributable to poor colon health. Colon cleansing is an important way to minimize the digestive tract's exposure to the multitude of micro-organisms encountered daily. Yet, relatively speaking, a properly functioning colon is actually quite clean compared to one that is filled with toxic substances, parasites, and pathogenic yeasts, fungi, and bacteria.

Larch Arabinogalactan is also believed to act as a prebiotic; it stimulates the colonic arowth of such bacteria as bifid bacteria and lactobacilli that confer certain health benefits. Ingestion of Larch Arabinogalactan has a significant effect on enhancing beneficial gut microflora, specifically increasing anaerobes such as Lactobacillus. Robinson R R, Feirtag J, Slavin J L, 20(4) J Am Coll Nutr 279-85 (2001).

Short chain fatty acids, primarily acetate, propionate, and butyrate, are produced in the colon by fermentation of dietary carbohydrates, particularly from degradation-resistant starches and dietary fiber, play an important role in intestinal health. These acids are the principal energy source for the colonic epithelial cells. The non-absorbed fiber of Arabinogalactan is easily fermented by the distal gut microflora, resulting in an elevated production of short-chain fatty acids, primarily butyrate, and, to a lesser extent, propionate.

Ammonia is produced as a by-product in the colon by bacterial fermentation of protein and Other nitrogen-containing substances. Research indicates that ammonia levels as low as 5 mmol/L can have detrimental effects on epithelial cells that line the colon. The toxicity of ammonia toward colonic epithelial cells can lead to cell destruction and increased turnover of these cells.

Many clinicians use prebiotics as a supplemental support for intestinal conditions including diverticulosis, leaky-gut, irritable bowel syndrome, as well as inflammatory bowel diseases such as Crohn's disease and ulcerative colitis. Studies have shown that Larch Arabinogalactan consumption reduces intestinal ammonia generation. Robinson, R R., Feirtag J., Slavin, Th., 20(4) J Am Coll Nutr 279-85 (2001). Since even low ammonia levels can have damaging effects on intestinal colonic cells, Larch Arabinogalactan can be supportive to patients who are unable to detoxify ammonia.

Larch Arabinogalactan has been isolated and characterized as a low molecular weight, 9 kDalton Arabinogalactan fragment and a purified Arabinogalactan, 37 kDa, from Larix occidentalis that is composed of repeating units of similar molecular weight and composition. A 9 kDa Arabinogalactan may be obtained in high yield from 37 kDa Arabinogalactan either by autoclaving at 121 degrees C. or by exposure to alkaline solution in the presence of sodium borohydride. The weight average molecular weight of 37 kDa Arabinogalactan was determined to be 37 and 38 kDa by intensity light scattering and sedimentation equilibrium, respectively. The weight average molecular weight of 9 k Da Arabinogalactan was determined to be 9.1 and 9.5 kDa by intensity light scattering and sedimentation equilibrium, respectively.

MALDI-TOF mass spectrometry yielded a molecular weight at the peak of the distribution of 8.3 kDa for Arabinogalactans (9 kDa). Both Arabinogalactans (37 kDa) and Arabinogalactans (9 kDa) exhibited narrow molecular-weight distributions (Mw/Mn approximately 1.2). Arabinogalactans (37 kDa) and Arabinogalactans (9 kDa) exhibit nearly identical 13C-NMR spectra, monosaccharide composition, and sugar linkages. It is proposed that Arabinogalactans (37 kDa) are composed of covalently bound subunits of Arabinogalactans (9 kDa). Arabinogalactans (37 kDa) and Arabinogalactans (9 kDa) bind isolated hepatocyte glycoprotein receptor equally well. As a result Arabinogalactans (9 kDa) is a candidate for use in hepatocyte directed drug delivery and may be more desirable for such use than is Arabinogalactans (37 kDa).

Larch Arabinogalactan in powder form is typically dosed in teaspoons or tablespoons at a concentration of approximately 3 grams per teaspoon. The adult dosage is one to three teaspoons per day in divided doses.

Because of its .mild taste and excellent solubility in water or juice, it is easy to use with children. Clinical feedback suggests an occasional. reaction of bloating and flatulence in less than three percent of individuals, usually females.

U.S. Pat. No. 4,950,751 to DeWitt, Jill E., and assigned to The Nanci Corporation International, describes a method of extracting galactans from a source including the steps of providing a quantity of solid particles of the source, forming a mixture of the particles and liquid extraction medium for galactans and recovering extracted galactans. The improvement is subjecting the mixture to an effective amount of sonic energy for enhancing the extraction of the galactans from the source particles.

U.S. Pat. No. 7,241,461 to Myhill, et. al., and assigned to Lifeline Nutraceuticals Corp., describes a composition having at least about 150 milligrams Bacopa monniera extract, wherein the Bacopa monniera extract has 45 percent bacosides and at least about 225 milligrams silybum marianum (milk thistle) extract. The milk thistle extract has between about 70 percent and about 80 percent silymarin, at least about 150 mg Withania somnifera (ashwagandha) powder and at least about 75 milligrams Camellia sinensis (green tea extract). The green tea extract has 98 percent polyphenols, the polyphenols consisting of 45 percent (−)-epigallocatechin gallate and at least 75 milligrams Curcuma longa (turmeric) extract. The turmeric extract has 95 percent curcumin wherein the composition increases the enzyme activity level of at least one antioxidant enzyme selected from the group consisting of: superoxide dismutase and catalase and decreases the plasma concentration level of thiobarbituric acid reactive chemical species, when administered in an effective amount to a mammalian subject in need thereof.

U.S. Pat. No. 5,756,098 to Price, et. al., and assigned to The University of Montana, Larex International Inc. and Crown Iron Works Co., describes a method of recovering an exudate having arabinogalactan, a pressed plant fiber product, and an extract liquor from a fibrous woody plant material. The .method compresses the fibrous woody plant material in absence of any added solvent to recover a liquid exudate having arabinogalactan from the fibrous woody plant material and a first pressed plant fiber product from the fibrous woody plant material and impregnating the first plant fiber product with a solvent to recover an extract liquor and impreenated plant fiber.

U.S. Pat. No. 7,288,374 to Pincemail, et. al., and assigned to Probiox, S A, describes a method for determining the relative amounts of oxidative stress markers in a group of individuals determined to have a risk factor for oxidative stress. The method providing measuring the amount of at least 10 different oxidative stress markers in a sample of whole blood or component thereof obtained from each individual of the group of individuals determined to have a risk factor for oxidative stress and comparing the amount of each of the oxidative stress markers in the group of individuals determined to have a risk factor for oxidative stress with the amount of each of the oxidative stress markers measured in a group of healthy individuals. The relative amounts of oxidative stress markers in the group of individuals determined to have a risk factor for oxidative stress relative to healthy individuals is determined here.

### SUMMARY

This inventor has discovered that the combination of the potent antioxidant, dihydroquercetin, with the powerful immune modulator Larch Arabinogalactan, is effective in interrupting the cascade of oxidative stress and metabolic inflammation that upregulates the expression of pro-inflammatory cytokines. In an embodiment of the disclosure the dietary supplement composition effective in quenching free oxygen radicals comprises:
Dihydroquercetin, also known as taxifolin;
Ascorbic acid, also known as Vitamin C; and
Arabinogalalctan, most specifically Larch Arabinogalactan of a low molecular weight.

The composition may be administered as a capsule, tablet, liquid, microsome, suppository or dermal patch and may include a pharmaceutically acceptable carrier.

### DETAILED DESCRIPTION OF THE PREFERRED EMBODIMENTS

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In the Summary above, the Description of the Invention, and the Claims and Abstract below, reference may be made to particular features (including method steps) of the invention. It is to he understood that this disclosure includes possible combinations of such particular features. For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

The term “comprises” and grammatical equivalents thereof are used herein to mean that other components, ingredients, steps etc. are optionally present. For example, an article “comprising” (or “which comprises”) components A, B and C can consist of (i.e. contain only) components A, B and C, or can contain not only components A, B and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number or the indefinite article “a” (meaning “one”) is used herein to denote the start of a range beginning with that number (which may be a range having an upper limit or no upper limit, depending on the variable being defined). For example “at least one” or “at least a” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. If, in this disclosure, a range is given as “(a first number) to (a second number)” or “(a first number)-(a second number)”, this means a range whose lower limit is the first number and whose upper limit is the second number. For example, 0-10 mm means a range whose lower limit is 0 mm, and whose upper limit is 10 mm.

The term “or” is used herein as a conjunction used to link alternatives in a series of alternatives. The term “and/or” is used herein as a conjunction meaning that either or both of two options may be valid.

In a specific embodiment the Arabinogalactan is low molecular weight Larch Arabinogalactan, LAG. The dihydroquercetin may be present in an amount ranging from about 4 mg to about 450 mg, the Vitamin C is present in an amount ranging from Ting to about 700 mg and the Arabinogalactans is present in an amount ranging from about 38 mg to about 3000 mg.

More specifically; the dihydroquercetin is present in an amount ranging from about 15 mg to about 150 mg, the Vitamin C is present in an amount ranging from 20 mg to about 200 mg and the Larch Arabinogalactan. is present in an amount ranging from about 12 mg to about 1500 mg. In its most specific embodiment, the dihydroquercetin is present in an amount of about 50 mg, the Vitamin C is present in an amount of about 70 mg and the Larch Arabinogalactan is present in an amount of about 500 mg.

The composition is effective at enhancing the immune modulating properties of Larch Arabinogalactan by quenching the free radicals that initiate the cascade of oxidative stress and inflammation, and may demonstrate anti-inflammatory and anti-allergic properties, or enhancement of immune function.

In an additional embodiment the quenching of free radicals by the composition further supports the flexibility of blood cells to navigate the microvasculature.

In another embodiment dihydroquercetin, in an ascorbic acid-ascorbate oxidase reaction mixture with Larch Arabinogalactan positively affected both the initial intensity of the ascorbate radical and extended the ascorbate radical lifetime.

In yet another embodiment quenching of free radicals by the composition further maintains healthy collagen and stimulates the body's production of collagen.

In an additional embodiment quenching of free radicals by the composition further supports capillary strength and helps to regulate capillary permeability.

in another embodiment the composition further scavenges reactive oxygen and nitrogen species (ROS-RNS) such as hydroxyl, peroxyl, superoxide, peroxynitrite, and nitroxide radicals as well as singlet oxygen and hypochlorite.

In another embodiment the quenching of free radicals by the composition further provides substantial protection to the eye against ultraviolet- generated free-radical damage.

In another embodiment the quenching of free radicals by the composition further provides antioxidant protection in neutrophils.

In another embodiment quenching of free radicals by the composition further provides antioxidant protection against ROS produced during phagocytosis.

In another embodiment the quenching of free radicals by the composition further provides antioxidant protection in semen, against oxidative damage to sperm DNA.

In another embodiment the quenching of free radicals by the composition protects further against LDL oxidation by scavenging ROS in the aqueous phase before they initiate cell membrane or fatty acid lipid peroxidation.

In another embodiment the quenching of free radicals by the composition further regenerates vitamin E back to its reduced state.

In another embodiment the Vitamin C is present as dihydroascorbic acid and more readily permeates the membranes of blood and intestinal cells after which dihydroascorbic acid, the oxidised form of vitamin. C, is reduced intracellularly back to ascorbic acid, the reduced. form of vitamin C.

In another embodiment the quenching of free radicals by the composition further enhances the vitamin C localization in the cytosol, accumulation intracellularly and in plasma.

This inventor has also discovered a method to enhance the immune modulating properties of Larch Arabinogalactan by quenching excessive production of free radicals that initiate the cascade of oxidative stress and inflammation by administering the composition comprising dihyroquercetin, vitamin C, and Arabinogalactan.

In specific embodiments, the method encompasses the various additional benefits of the quenching of free. radicals. The quenching of free radicals is beneficial because it increases the flexibility of blood cells; maintains healthy collagen and stimulates the production of said collagen; supports capillary strength and regulates capillary permeability; provides substantial antioxidant protection against ultraviolet generated free-radical damage to the eye; provides substantial antioxidant protection to neutrophils; provides substantial antioxidant protection against ROS produced during phagocytosis; provides substantial antioxidant protection for semen by providing oxidative damage to sperm DNA; protects against LDL oxidation by scavenging ROS in the aqueous phase before peroxidation of cell membranes or fatty acid lipids; regenerates vitamin E back to its reduced form; assists significantly in vitamin C localization in cytosol and provides intracellular accumulation in plasma.

In yet another embodiment, the method is taught for modulation of the immune system by administering the composition to a mammal in need thereof, most specifically a human. The composition exhibits immune stimulatory properties, and the composition may be used to control inflammation, and allergic reaction.

Also taught are methods to administer the Vitamin C in an ascorbic acid-ascorbate oxidase reaction mixture that increases the initial intensity of the ascorbate radical and the lifetime of said ascorbate radical.

In yet another embodiment a method is disclosed to quench both reactive oxygen species and reactive nitrogen species, including hydroxyl radical, peroxyl radical, superoxide radical, peroxynitrite radical, nitroxide radicals, singlet oxygen, and hypochlorite.

In yet another embodiment, the enhancement of the immune stimulatory properties of the Larch Arabinogalactan is used in methods of modulating the immune system by administering the composition of claim 6 to a mammal in need thereof. This method may be used to control inflammation and allergic reaction, or to enhance immune function.

In yet another embodiment, a method is disclosed wherein the vitamin C is present in the form of dihydroascorbic acid, and more readily permeates the membranes of blood and intestinal cells after which dihydroascorbic acid is reduced intracellularly to ascorbic acid.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

1. A dietary supplement composition, comprising:
a dihydroquercetin;
a vitamin C; and
an arabinogalactan.

2. The dietary supplement composition of claim 1, wherein said arabinogalactan is present as a larch arabinogalactan.

3. The dietary supplement composition of claim 2, wherein said larch arabinogalactan has a molecular weight between about 9.1 kDas and about 37 kDas.

4. The dietary supplement composition of claim 2 wherein the composition is formulated to be administered as a capsule, a tablet, a liquid, a microsome, a suppository or adermal patch.

5. The dietary supplement composition of claim 3, further comprising a pharmaceutically acceptable carrier.

6. The dietary supplement composition of claim 2, wherein said composition is effective to enhance immune modulating properties of said larch arabinogalactan by quenching free radicals that initiate a cascade of oxidative stress and inflammation.

7. The dietary supplement composition of claim 6, wherein the dihydroquercetin and larch arabinogalactan are extracted from saw logs and a bark of Siberian and Dahurian Larch.

8. The dietary supplement composition of claim 6, wherein the dihydroquercetin is present in an amount ranging from about 4 mg to about 450 mg, the vitamin C is present in an amount ranging from 7 mg to about 700 mg and the larch arabinogalactan is present in an amount ranging from about 38 mg to about 3000 mg.

9. The dietary supplement composition of claim 8, wherein the dihydroquercetin is present in an amount ranging from about 15 mg to about 150 mg, the vitamin C is present in an amount ranging from 20 mg to about 200 mg and the larch arabinogalactan is present in an amount ranging about 12 mg to about 1500 mg.

10. The dietary supplement composition of claim 9, wherein the dihydroquercetin is present in an amount of about 50 mg, the vitamin C is present in an amount of about 70 mg and the larch arabinogalactan is present in an amount of about 50 mg.

11. The dietary supplement composition of claim 6, wherein said quenching of free radicals increases flexibility of blood cells.

12. The dietary supplement composition of claim 6, wherein said quenching of free radicals keeps collagen healthy and stimulates the human body production of said collagen.

13. The dietary supplement composition of claim 6, wherein said quenching of free radicals supports capillary strength and regulates capillary permeability.

14. The dietary supplement composition of claim 6, wherein said quenching of free radicals is effective to scavenge reactive nitrogen species.

15. The dietary supplement composition of claim 6, wherein said quenching of free radicals is effective to guard an eye against ultraviolet generated free-radical damage.

16. The dietary supplement composition of claim 6, wherein said quenching of free radicals is effective to provide a substantial antioxidant protection in neutrophils.

17. The dietary supplement composition of claim 6, wherein said quenching of free radicals is effective to provide substantial antioxidant protection against ROS produced during phagocytosis.

18. The dietary supplement composition of claim 6, wherein said quenching of free radicals is effective to provide substantial antioxidant protection for semen by preventing oxidative damage to sperm DNA.

19. The dietary supplement composition of claim 6, wherein said quenching of free radicals is effective to protect against LDL oxidation by scavenging ROS in the aqueous phase before peroxidation of cell membranes or fatty acid lipids.

20. The dietary supplement composition of claim 6, wherein said quenching of free radicals is effective to regenerate vitamin E back to its active state.

21. The dietary supplement composition of claim 6, wherein said vitamin C is present in a form of a dihydroascorbic acid and more readily permeates membranes of blood and intestinal cells after which said dihydroascorbic acid is reduced intracellularly back to an ascorbic acid, i.e., a reduced form of vitamin C.

22. The dietary supplement composition of claim 6, wherein said quenching of free radicals is effective to assist significantly in vitamin C localization in cytosol and to provide intracellular accumulation in plasma.

23-40. (canceled)
